(19) United States
(12) Patent Application Publication
(10) Pub. No.: US 2023/0115828 A1
(43) Pub. Date: Apr. 13, 2023

(54) GENOMIC CLASSIFIERS FOR PROGNOSING AND TREATING CLINICALLY AGGRESSIVE LUMINAL BLADDER CANCER

(71) Applicant: Decipher Biosciences, Inc., San Diego, CA (US)

(72) Inventors: Joep Jacobus de Jong, Rotterdam (NL); Yang Liu, Vancouver (CA); Ewan A. Gibb, Vancouver (CA); Elai Davicioni, La Jolla, CA (US)

(21) Appl. No.: 17/915,646

(22) PCT Filed: Mar. 31, 2021
(86) PCT No.: PCT/US2021/025090

§ 371 Date: Sep. 29, 2022

Related U.S. Application Data
(60) Provisional application No. 63/004,732, filed on Apr. 3, 2020.

(51) Int. Cl. C12Q 1/6886; G01N 33/574

(52) U.S. Cl. CPC C12Q 1/6886; G01N 33/57407; C12Q 2600/118; C12Q 2600/158; C12Q 2600/106

(57) ABSTRACT

The present disclosure pertains to the field of personalized medicine and methods for prognosing and treating bladder cancer. In particular, the disclosure relates to the use of genomic classifiers and genomic signatures for the prognosis and/or treatment of individuals with bladder cancer. The present disclosure provides methods for subtyping bladder cancer. The present disclosure also provides methods and compositions for treating bladder cancer.

### CROSS-REFERENCE TO RELATED APPLICATIONS

This application claims priority to U.S. Ser. No. 63/004,732, filed Apr. 3, 2020, which is herein incorporated by reference in its entirety.

### FIELD OF THE INVENTION

The present disclosure pertains to the field of personalized medicine and methods for prognosing and treating bladder cancer. In particular, the disclosure relates to the use of genomic classifiers and genomic signatures for the prognosis and/or treatment of individuals with bladder cancer. The present disclosure provides methods for subtyping bladder cancer. The present disclosure also provides methods and compositions for treating bladder cancer.

### BACKGROUND OF THE INVENTION

Bladder cancer has a global annual incidence of 430,000 patients, making it the fourth and tenth most common malignancy in men and women, respectively. A current limitation in the treatment of patients with high-risk non-muscle invasive and muscle-invasive bladder cancer (MIBC) is the poor accuracy of clinical staging. About 40% of patients with a diagnosis of localized cT1-2N0M0 bladder cancer experience pathological upstaging at radical cystectomy (RC), defined as the presence of non-organ confined (NOC; ≥pT3 and/or pN+) disease. (Shariat et al. Eur Urol, 51: 137, 2007.) Pathological upstaging from pre-cystectomy clinical stage ≤T2 disease to NOC correlates with an increased risk of cancer-specific mortality (CSM). (Shariat et al. Eur Urol, 51: 137, 2007.) While patients with organ confined (OC) disease may be cured by cystectomy alone, patients with NOC disease are likely to benefit most from neoadjuvant chemotherapy (NAC). (Shariat et al. Clin Cancer Res, 12: 6663, 2006; Stein et al. J Clin Oncol, 19: 666, 2001; and Grossman et al. N Engl J Med, 349: 859, 2003.) However, patients with cT1 are not currently offered NAC a significant proportion of these patients have NOC and could benefit from this treatment if accurate staging were available.

Molecular subtyping has been developed for predicting therapeutic benefit, patient outcome and, more recently, identifying patients with NOC at clinical staging using transurethral resected bladder tumor (TURBT) tissue. (Lotan et al. Eur Urol, 76: 200, 2019.) For patients with cT1-T2 disease, luminal tumors had a significantly lower risk of pathological upstaging to NOC disease than non-luminal (luminal-infiltrated, basal or claudin-low) tumors. (Lotan et al. Eur Urol, 76: 200, 2019.) Luminal tumors have also been reported to have a less aggressive clinical behavior and may benefit less from NAC. (Seiler et al. Eur Urol, 72: 544, 2017.) However, as molecular subtyping advances, additional luminal subtypes have been described suggesting that heterogeneity exists within this subtype. (Robertson et al. Cell, 171: 540, 2017; Kamoun et al. Eur Urol, 2019; and Bernardo et al. J Pathol, 249: 308, 2019.)

Although luminal tumors have lower rates of NOC disease than non-luminal tumors, approximately one-third of luminal cT1-2 patients are upstaged to NOC disease at RC. (Lotan et al. Eur Urol, 76: 200, 2019.) This subgroup of patients with luminal tumors might have aggressive disease, and therefore they are candidates for neoadjuvant therapies, including platinum-based NAC. If these patients could accurately be identified at the time of diagnosis, we could stratify luminal patients to the best treatment option for their stage of disease. Thus, there is a need in the art for improved methods for subtyping bladder cancer to identify clinically aggressive luminal bladder tumors with non-organ confined disease at radical cystectomy.

This background information is provided for the purpose of making known information believed by the applicant to be of possible relevance to the present disclosure. No admission is necessarily intended, nor should be construed, that any of the preceding information constitutes prior art against the present disclosure.

### SUMMARY OF THE INVENTION

The present disclosure relates to methods, systems and kits for the diagnosis, prognosis, and treatment of bladder cancer in a subject. The disclosure also provides biomarker targets and genomic classifiers that are clinically useful for identifying bladder cancer subjects that are at high risk of upstaging to non-organ confined disease at radical cystectomy. The disclosure further provides biomarker targets and genomic classifiers that define subgroups of bladder cancer, clinically useful classifiers for distinguishing bladder cancer subtypes, bioinformatic methods for determining clinically useful classifiers, and methods of use of each of the foregoing. The methods, systems and kits can provide expression-based analysis of biomarker targets for purposes of prognosing bladder cancer in a subject. Further disclosed herein, are probe sets for use in prognosing bladder cancer in a subject. Genomic classifiers for prognosing a bladder cancer and methods of treating bladder cancer based on the expression level of biomarker targets of the disclosure are also provided.

An aspect of the disclosure relates to a method comprising providing a biological sample from a subject having bladder cancer; and detecting the presence or expression level of a plurality of targets in the sample wherein the plurality of targets is selected from Table 3 and/or Table 4. In an embodiment, the method further comprises prognosing the bladder cancer according to a genomic classifier based on the expression level of the plurality of targets. In an embodiment, the prognosing is upstaging to non-organ confined cancer. In an embodiment, the methods further comprise subtyping the bladder cancer to a luminal subtype. In an embodiment the bladder cancer is a luminal subtype. In an embodiment the methods further comprise determining that the subject has a favorable prognosis if the expression levels of the plurality of targets indicate that the subject will not have non-organ confined tumors or determining that the subject has an unfavorable prognosis if the expression levels of the plurality of targets indicate that the subject will have non-organ confined tumors. In an embodiment the methods further comprise determining that the subject has a less aggressive tumor if the expression levels of the plurality of targets indicate that the subject will not have non-organ confined tumors or determining that the subject has a more aggressive tumor if the expression levels of the plurality of targets indicate that the subject will have non-organ confined tumors. In an embodiment, the methods further comprise administering neoadjuvant chemotherapy to the subject if the subtyping indicates that the subject has the luminal-papillary subtype and administering neoadjuvant chemotherapy to the subject if the subtyping indicates that the subject has the basal/squamous, luminal, luminal-infiltrated, or neuronal subtype. In an embodiment the neoadjuvant chemotherapy comprises administering cisplatin. In an embodiment the methods are performed prior to treatment of the patient with anti-cancer therapy. In an embodiment, the biological sample is a biopsy. In an embodiment the biological sample is a urine sample, a blood sample, or a bladder tumor sample. In an embodiment, the biological sample is transurethral resected bladder tumor tissue. In an embodiment the subject is a human being. In an embodiment the level of expression is increased or reduced compared to a control. In an embodiment detecting the presence or level of expression comprises performing in situ hybridization, a PCR-based method, a sequencing method, an array-based method, an immunohistochemical method, an RNA assay method, or an immunoassay method. In an embodiment the detecting the presence or level of expression comprises using a reagent selected from the group consisting of a nucleic acid probe, one or more nucleic acid primers, and an antibody. In an embodiment, detecting the presence or the level of expression comprises detecting the presence or level of an RNA transcript. In an embodiment the plurality of targets comprises the 99 genes in Table 4.

An aspect of the disclosure relates to a method for determining a treatment for a subject who has bladder cancer, the method comprising: providing a biological sample from the subject; a) detecting the presence or expression level in the biological sample for a plurality of targets selected from Table 3 and/or Table 4; b) prognosing the bladder cancer of the subject according to a genomic classifier based on the levels of expression of the plurality of genes; and c) determining whether or not the subject is likely to be responsive to neoadjuvant chemotherapy based on the expression levels of the plurality of targets in the sample; and d) prescribing neoadjuvant chemotherapy to the subject if the patient is identified as likely to be responsive to neoadjuvant chemotherapy. In an embodiment the neoadjuvant chemotherapy is cisplatin.

An aspect of the disclosure relates to a kit for prognosing bladder cancer in a subject, the kit comprising agents for detecting the presence or expression levels for a plurality of targets, wherein said plurality of genes comprises one or more targets selected from Table 3 and/or Table 4. In an embodiment, the agents comprise reagents for performing in situ hybridization, a PCR-based method, an array-based method, a sequencing method, an immunohistochemical method, an RNA assay method, or an immunoassay method. In an embodiment, the agents comprise one or more of a microarray, a nucleic acid probe, a nucleic acid primer, or an antibody. In an embodiment, the kit comprises at least one set of PCR primers capable of amplifying a nucleic acid comprising a sequence of a gene selected from Table 3 and/or Table 4 or its complement. In an embodiment, the kit comprises at least one probe capable of hybridizing to a nucleic acid comprising a sequence of a gene selected from Table 3 and/or Table 4 or its complement. In an embodiment, the kit further comprises information, in electronic or paper form, comprising instructions on how to determine if a subject is likely to be responsive to neoadjuvant chemotherapy. In an embodiment, the kit further comprises one or more control reference samples. In an embodiment, the disclosure provides a kit for prognosing bladder cancer comprising a probe set for prognosing bladder cancer in a subject, the probe set comprising a plurality of probes for detecting a plurality of target nucleic acids, wherein the plurality of target nucleic acids comprises one or more gene sequences, or complements thereof, of genes selected from Table 3 and/or Table 4. In an embodiment, at least one probe is detectably labeled. In an embodiment, the kit further comprises a computer model or algorithm for designating a treatment modality for the subject. In an embodiment, the kit further comprises a computer model or algorithm for normalizing the expression level or expression profile of the plurality of target nucleic acids.

An aspect of the disclosure relates to a probe set for prognosing bladder cancer in a subject, the probe set comprising a plurality of probes for detecting a plurality of target nucleic acids, wherein the plurality of target nucleic acids comprises one or more gene sequences, or complements thereof, of genes selected from Table 3 and/or Table 4. In an embodiment, at least one probe is detectably labeled.

An aspect of the disclosure relates to a system for analyzing a bladder cancer, the system comprising: a) a probe set for prognosing bladder cancer in a subject, the probe set comprising a plurality of probes for detecting a plurality of target nucleic acids, wherein the plurality of target nucleic acids comprises one or more gene sequences, or complements thereof, of genes selected from Table 3 and/or Table 4; and b) a computer model or algorithm for analyzing an expression level or expression profile of the plurality of target nucleic acids hybridized to the plurality of probes in a biological sample from a subject who has bladder cancer and prognosing the bladder cancer of the subject according to a genomic classifier based on the expression level or expression profile of the target nucleic acids in the sample.

An aspect of the disclosure relates to a method for treating a subject with bladder cancer, the method comprising: a) providing a biological sample from a subject having bladder cancer; b) detecting the presence or expression level in the biological sample for a plurality of targets selected from Table 3 and/or Table 4; and c) administering a treatment to the subject, wherein the treatment is selected from the group consisting of neoadjuvant chemotherapy or an anti-cancer treatment. In an embodiment, the anti-cancer treatment is selected from the group consisting of surgery, chemotherapy, radiation therapy, immunotherapy, biological therapy, hormonal therapy, and photodynamic therapy. In an embodiment, the method further comprises prognosing the bladder cancer in the subject according to a genomic classifier based on the presence or expression levels of the plurality of targets, wherein said prognosing comprises determining whether or not the subject is likely to have non-organ confined tumors based on the levels of expression of the plurality of targets in the sample. In an embodiment, the method further comprises prognosing the bladder cancer in the subject according to a genomic classifier based on the presence or expression levels of the plurality of targets, wherein said prognosing comprises determining whether or not the subject is likely to be responsive to neoadjuvant chemotherapy based on the levels of expression of the plurality of genes in the sample.

An aspect of the disclosure relates to methods for prognosing and treating bladder cancer in a subject. In an embodiment, the disclosure provides a method comprising providing a biological sample from a subject having bladder cancer; and detecting the presence or expression level of a plurality of targets in the sample wherein the plurality of targets is selected from Table 3 or Table 4. In an embodiment, the plurality of targets are selected from the group consisting of ADAM17, APOC1, ARL17A, ARL8B, ASNS, BDH2, BHLHE40, BRK1, C3orf14, C4orf46, CD151, CD9, CERS2, CH17-140K24.5, CISD2, CMTM6, CMTM7, CNN3, CRIPAK, CSRP2, CST6, CTB-102L5.4, CTD-2003C8.2, CTD-2547L24.4, CTD-3220F14.1, CYP4Z1, DOCK3, ENSA, FAM25G, FO538757.2, FZD4, GARS, GTF3A, HAR1B, HDAC2, HMGB2, HMGN4, HNRNPAB, KCTD11, KRT14, KRT17, KRTAP13-3, LINC00960, MCL1, MKRN2OS, MLLT11, MTERF3, MTMR11, MYO10, NAA50, OR10G9, P2RY2, PDIA6, POLB, PPAPDC2, PRDX1, PSMD4, PVRIG, RARRES1, RP11-105N13.4, RP11-1082L8.3, RP11-115H18.1, RP11-145M4.3, RP11-168F9.2, RP11-247A12.7, RP11-285F16.1, RP11-38L15.8, RP11-462G22.2, RP11-468N14.13, RP11-484P15.1, RP11-539L10.3, RP11-74E22.6, RP11-876F14.1, RP3-406A7.7, RP5-827L5.2, RP5-855F16.1, SERP1, SF3B5, SFN, SH3PXD2B, SLC2A1, SLC4A7, SLC6A9, SPG20, SPIDR, SPOCK1, SREBF1, ST6GALNAC2, TBC1D2, TCEAL1, TCEAL3, TM4SF1, TMEM40, TRTM16L, TRIO, UCHL3, VDAC3, VHL, WBP5, WDR45B, WLS, YWHAG, YWHAQ, ZNF256, ZNF260, ZNF662, ZNF680, ZNF717, and ZNF790. In other embodiments In an embodiment, the plurality of targets are selected from the group consisting of AC005477.1, AC005614.5, AC011516.1, AC011525.2, AC104653.1, AC112715.2, ACBD7, AL022578.1, ANP32D, CCND2, CGB1, CLC, CTA-212A2.3, CTA-276F8.1, CTA-384D8.33, CTA-481E9.3, CTC-264010.2, CTC-471C19.2, CTD-203414.2, FAM102B, FAM63B, GLCE, HIF1A, HIGD1C, HIST2H2BE, HNMT, KANTR, KIAA1551, LAPTM5, LNC00350, LINCO1017, LINC01288, LL22NC03-102D1.18, LLNLR-246C6.1, MGAT2, MRPL20, MYADM, NHLRC1, OR10A2, OR13F1, OR13H1, OR52W1, ORC2, PCEDIB-AS1, PCNXL4, PRR32, PVALB, RP1-90G24.6, RP11-1030E3.1, RP11-164C1.2, RP11-173D3.1, RP11-18C24.8, RP11-23F23.2, RP11-25I9.3, RP11-265E18.1, RP11-265N7.1, RP11-284F21.11, RP11-347C12.10, RP11-353N14.4, RP11-368L12.1, RP11-379L18.3, RP11-381K20.4, RP11-435J9.2, RP11-440I14.2, RP11-44D19.1, RP11-467L19.16, RP11-48611.2, RP11-554A11.7, RP11-571L19.8, RP11-612B6.1, RP11-63A1.2, RP11-662J14.1, RP11-70F11.7, RP11-789C2.1, RP11-78J21.4, RP11-7F17.4, RP11-810P12.5, RP3-395M20.12, ST20, ST3GAL5-AS1, TAAR2, and TBC1D30. In an embodiment, the plurality of targets are selected from the group consisting of ADAM17, APOC1, ARL17A, ARL8B, BHLHE40, BRK1, CD9, CERS2, CH17-140K24.5, CISD2, CMTM6, CMTM7, CNN3, CRIPAK, CSRP2, CST6, CTB-102L5.4, CTD-2003C8.2, CTD-2547L24.4, CTD-3220F14.1, CYP4Z1, ENSA, FO538757.2, GARS, GTF3A, HMGB2, HNRNPAB, KRT14, KRT17, MCL1, MKRN2OS, MLLT11, MTERF3, NAA50, OR10G9, PDIA6, POLB, PRDX1, PSMD4, RARRES1, RP11-105N13.4, RP11-168F9.2, RP11-247A12.7, RP11-38L15.8, RP11-462G22.2, RP11-539L10.3, RP11-74E22.6, RP11-876F14.1, RP3-406A7.7, RP5-827L5.2, SF3B5, SFN, SLC2A1, SPOCK1, ST6GALNAC2, TCEAL1, TCEAL3, TM4SF1, TMEM40, UCHL3, VDAC3, WBP5, WDR45B, WLS, YWHAG, YWHAQ, ZNF256, ZNF260, ANP32D, CTA-276F8.1, CTA-384D8.33, CTD-2034I4.2, FAM63B, HIF1A, HIST2H2BE, HNMT, KIAA1551, LAPTM5, LL22NC03-102D1.18, MRPL20, MYADM, OR13H1, OR52W1, PVALB, RP1-90G24.6, RP11-173D3.1, RP11-18C24.8, RP11-368L12.1, RP11-379L18.3, RP11-381K20.4, RP11-435J9.2, RP11-440I14.2, RP11-467L19.16, RP11-486I11.2, RP11-554A11.7, RP11-571L19.8, RP11-612B6.1, RP11-789C2.1, and ST20.

An aspect of the disclosure relates to evaluating the significance of the expression levels of one or more biomarker targets of the disclosure using, for example, a T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) or Multivariable Analysis Hazard Ratio P-value (mvaHRPval).

These and other embodiments of the subject disclosure will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference. Further, all publications, patents, and patent applications mentioned in this specification are also herein incorporated by reference for the particular subject matter referenced herein to the same extent as if each individual publication, patent, or patent application was specifically and individually indicated to be incorporated by reference.

### BRIEF DESCRIPTION OF THE DRAWINGS

FIG. 1 shows an embodiment of differential gene expression analysis comparing NOC and OC disease within the training set (N=75).

FIGS. 2A-2B show performance of an embodiment of the Luminal Upstaging Classifier (LUC) for predicting non-organ confined (pT3+ or pTanyN+) disease at radical cystectomy in luminal urothelial carcinoma. For the (A) training set (n=75) and (B) testing set (n=25), the predicted probabilities are shown with box plots with the computed cutpoint of 0.45. The right panels show the sensitivity and specificity of the model.

FIGS. 3A-3B show density plots of an embodiment of the predicted Luminal Upstaging Classifier probability within the training (A) (N=75) and testing (B) (N=25) sets of the luminal MOL cohort.

FIGS. 4A-4B show an embodiment of Kaplan-Meier curves for overall survival of (A) urothelial carcinoma patients with organ confined (OC) versus non-organ confined disease (NOC) at radical cystectomy (RC) and (B) Luminal Upstaging Classifier (LUC) at the diagnostic transurethral resection of a bladder tumor (TURBT).

FIGS. 5A-5B show an embodiment of overall survival of Luminal Upstaging Classifier positive (LUC+) cases in training (A) and testing (B) sets.

FIG. 6 shows an embodiment of overall survival of Luminal Upstaging Classifier (LUC) false positive (FP) cases in luminal MOL cohort.

FIG. 7 shows an embodiment of Kaplan-Meier curves for overall survival of luminal urothelial carcinoma patients (n=83, cT2-T4) stratified by the Luminal Upstaging Classifier (LUC) at radical cystectomy in TCGA cohort.

FIG. 8 shows an embodiment of Kaplan-Meier curves for overall survival of N=35 luminal urothelial carcinoma patients stratified by the Luminal Upstaging Classifier (LUC) at TURBT in the NAC cohort.

### DETAILED DESCRIPTION OF THE INVENTION

The present disclosure relates to systems and methods for prognosing, subtyping, diagnosing, predicting, and/or monitoring the status or outcome of bladder cancer in a subject using expression-based analysis of a plurality of targets. Generally, the method comprises (a) optionally providing a sample from a subject; (b) assaying the expression level of a plurality of targets in the sample; and (c) prognosing, subtyping, diagnosing, predicting and/or monitoring the status or outcome of a bladder cancer based on the expression level of the plurality of targets. Assaying the expression level for a plurality of targets in the sample may comprise applying the sample to a microarray. In some instances, assaying the expression level may comprise the use of an algorithm. The algorithm may be used to produce a classifier. Alternatively, the classifier may provide a probe selection region. In some instances, assaying the expression level for a plurality of targets comprises detecting and/or quantifying the plurality of targets. In an embodiment, assaying the expression level for a plurality of targets comprises sequencing the plurality of targets. In an embodiment, assaying the expression level for a plurality of targets comprises amplifying the plurality of targets. In an embodiment, assaying the expression level for a plurality of targets comprises quantifying the plurality of targets. In an embodiment, assaying the expression level for a plurality of targets comprises conducting a multiplexed reaction on the plurality of targets. In some instances, the plurality of targets comprises one or more targets selected from Table 3 and/or Table 4. In some instances, the plurality of targets comprises at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, or at least about 99 targets selected from Table 3 and/or Table 4.

Further disclosed herein are methods for prognosing bladder cancer. Generally, the method comprises: (a) providing a sample from a subject: (b) assaying the expression level for a plurality of targets in the sample; and (c) prognosing the bladder cancer based on the expression level of the plurality of targets. In some instances, the plurality of targets comprises one or more targets selected from Table 3 and/or Table 4. In an embodiment, the biological sample comprises bladder cancer cells. In an embodiment, the biological sample comprises nucleic acids (e.g., RNA or DNA). In some instances, the plurality of targets comprises at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, or at least about 99 targets selected from Table 3 and/or Table 4. In some instances, prognosing the bladder cancer comprises determining whether the cancer would respond to an anti-cancer therapy. Alternatively, prognosing the bladder cancer comprises identifying the cancer as non-responsive to an anti-cancer therapy. Optionally, prognosing the bladder cancer comprises identifying the cancer as responsive to an anti-cancer therapy. In an embodiment, prognosing the bladder cancer comprises identifying subjects that are at high risk of upstaging to non-organ confined disease at radical cystectomy.

Before embodiments of the present disclosure are described in further detail, it is to be understood that this disclosure is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present disclosure.

### Targets

In some instances, assaying the expression level of a plurality of genes comprises detecting and/or quantifying a plurality of target analytes. In an embodiment, assaying the expression level of a plurality of genes comprises sequencing a plurality of target nucleic acids. In an embodiment, assaying the expression level of a plurality of biomarker genes comprises amplifying a plurality of target nucleic acids. In an embodiment, assaying the expression level of a plurality of biomarker genes comprises conducting a multiplexed reaction on a plurality of target analytes.

The methods disclosed herein often comprise assaying the expression level of a plurality of targets. The plurality of targets may comprise coding targets and/or non-coding targets of a protein-coding gene or a non-protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non-protein-coding gene structure may comprise an exon and intron. Usually, the exon region of a non-protein-coding gene primarily contains a UTR. The non-protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets comprises one or more targets selected from Table 3 and/or Table 4. In some instances, the plurality of targets comprises at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, at least about 99 targets selected from Table 3 and/or Table 4.

In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non-protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15, 20, 25, 30, 35, 40, 45, 50, 60, 70, 80, 90, 99 or more targets selected from a Random Forest (RF) classifier. The RF classifier may be an RF2, and RF3, or an RF4 classifier. The RF classifier may be an RF22 classifier (e.g., a Random Forest classifier with 22 targets). For example, a RF classifier of the present disclosure may comprise two or more targets selected from Table 3 and/or Table 4.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60, 70, 80, 90, 99 or more targets selected from an SVM classifier. The SVM classifier may be an SVM2 classifier. A SVM classifier of the present disclosure may comprise two or more targets selected from Table 3 and/or Table 4.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50, 53, 55, 57, 60 or more targets selected from a KNN classifier. The plurality of targets may comprise 65, 70, 75, 80, 85, 90, 95, 100 or more targets selected from a KNN classifier. For example, a KNN classifier of the present disclosure may comprise two or more targets selected from Table 3 and/or Table 4.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50, 53, 55, 57, 60 or more targets selected from a NB classifier. The plurality of targets may comprise 65, 70, 75, 80, 85, 90, 95, 100 or more targets selected from a NB classifier. For example, a NB classifier of the present disclosure may comprise two or more targets selected from Table 3 and/or Table 4.

The plurality of targets may comprise one or more targets selected from a recursive partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50, 53, 55, 57, 60 or more targets selected from an Rpart classifier. The plurality of targets may comprise 65, 70, 75, 80, 85, 90, 95, 100 or more targets selected from an Rpart classifier. For example, an Rpart classifier of the present disclosure may comprise two or more targets selected from Table 3 and/or Table 4.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15, 20, 30, 40, 50, 60, 70, 80, 90, 99 or more targets selected from a high dimensional discriminate analysis (HDDA) classifier. For example, an Rpart classifier of the present disclosure may comprise two or more targets selected from Table 3 and/or Table 4.

In an embodiment, the plurality of targets are selected from the group consisting of ADAM17, APOC1, ARL17A, ARL8B, ASNS, BDH2, BHLHE40, BRK1, C3orf14, C4orf46, CD151, CD9, CERS2, CH17-140K24.5, CISD2, CMTM6, CMTM7, CNN3, CRIPAK, CSRP2, CST6, CTB-102L5.4, CTD-2003C8.2, CTD-2547L24.4, CTD-3220F14.1, CYP4Z1, DOCK3, ENSA, FAM25G, FO538757.2, FZD4, GARS, GTF3A, HAR1B, HDAC2, HMGB2, HMGN4, HNRNPAB, KCTD11, KRT14, KRT17, KRTAPI3-3, LINC00960, MCL1, MKRN2OS, MLLT11, MTERF3, MTMR11, MYO10, NAA50, OR10G9, P2RY2, PDIA6, POLB, PPAPDC2, PRDX1, PSMD4, PVRIG, RARRES1, RP11-105N13.4, RP11-1082L8.3, RP11-115H18.1, RP11-145M4.3, RP11-168F9.2, RP11-247A12.7, RP11-285F16.1, RP11-38L15.8, RP11-462G22.2, RP11-468N14.13, RP11-484P15.1, RP11-539L10.3, RP11-74E22.6, RP11-876F14.1, RP3-406A7.7, RP5-827L5.2, RP5-855F16.1, SERP1, SF3B5, SFN, SH3PXD2B, SLC2A1, SLC4A7, SLC6A9, SPG20, SPIDR, SPOCK1, SREBF1, ST6GALNAC2, TBC1D2, TCEAL1, TCEAL3, TM4SF1, TMEM40, TRIM16L, TRIO, UCHL3, VDAC3, VHL, WBP5, WDR45B, WLS, YWHAG, YWHAQ, ZNF256, ZNF260, ZNF662, ZNF680, ZNF717, and ZNF790. In other embodiments In an embodiment, the plurality of targets are selected from the group consisting of AC005477.1, AC005614.5, AC011516.1, AC011525.2, AC104653.1, AC112715.2, ACBD7, AL022578.1, ANP32D, CCND2, CGB1, CLC, CTA-212A2.3, CTA-276F8.1, CTA-384D8.33, CTA-481E9.3, CTC-264010.2, CTC-471C19.2, CTD-203414.2, FAM102B, FAM63B, GLCE, HIF1A, HIGD1C, HIST2H2BE, HNMT, KANTR, KIAA1551, LAPTM5, LINC00350, LINC01017, LINC01288, LL22NC03-102D1.18, LLNLR-246C6.1, MGAT2, MRPL20, MYADM, NHLRC1, OR10A2, OR13F1, OR13H1, OR52W1, ORC2, PCED1B-AS1, PCNXL4, PRR32, PVALB, RP1-90G24.6, RP11-1030E3.1, RP11-164C1.2, RP11-173D3.1, RP11-18C24.8, RP11-23F23.2, RP11-2519.3, RP11-265E18.1, RP11-265N7.1, RP11-284F21.11, RP11-347C12.10, RP11-353N14.4, RP11-368L12.1, RP11-379L18.3, RP11-381K20.4, RP11-435J9.2, RP11-440I14.2, RP11-44D19.1, RP11-467L19.16, RP11-486I11.2, RP11-554A11.7, RP11-571L19.8, RP11-612B6.1, RP11-63A1.2, RP11-662J14.1, RP11-70F11.7, RP11-789C2.1, RP11-78J21.4, RP11-7F17.4, RP11-810P12.5, RP3-395M20.12, ST20, ST3GAL5-AS1, TAAR2, and TBCID30. In an embodiment, the plurality of targets are selected from the group consisting of ADAM17, APOC1, ARL17A, ARL8B, BHLHE40, BRK1, CD9, CERS2, CH17-140K24.5, CISD2, CMTM6, CMTM7, CNN3, CRIPAK, CSRP2, CST6, CTB-102L5.4, CTD-2003C8.2, CTD-2547L24.4, CTD-3220F14.1, CYP4Z1, ENSA, FO538757.2, GARS, GTF3A, HMGB2, HNRNPAB, KRT14, KRT17, MCL1, MKRN2OS, MLLT11, MTERF3, NAA50, OR10G9, PDIA6, POLB, PRDX1, PSMD4, RARRES1, RP11-105N13.4, RP11-168F9.2, RP11-247A12.7, RP11-38L15.8, RP11-462G22.2, RP11-539L10.3, RP11-74E22.6, RP11-876F14.1, RP3-406A7.7, RP5-827L5.2, SF3B5, SFN, SLC2A1, SPOCK1, ST6GALNAC2, TCEAL1, TCEAL3, TM4SF1, TMEM40, UCHL3, VDAC3, WBP5, WDR45B, WLS, YWHAG, YWHAQ, ZNF256, ZNF260, ANP32D, CTA-276F8.1, CTA-384D8.33, CTD-2034I4.2, FAM63B, HIF1A, HIST2H2BE, HNMT, KIAA1551, LAPTM5, LL22NC03-102D1.18, MRPL20, MYADM, OR13H1, OR52W1, PVALB, RP1-90G24.6, RP11-173D3.1, RP11-18C24.8, RP11-368L12.1, RP11-379L18.3, RP11-381K20.4, RP11-435J9.2, RP11-440114.2, RP11-467L19.16, RP11-486I11.2, RP11-554A11.7, RP11-571L19.8, RP11-612B6.1, RP11-789C2.1, and ST20.

### Probes/Primers

The present disclosure provides a probe set for subtyping and/or prognosing, diagnosing, monitoring and/or predicting a status or outcome of bladder cancer in a subject, the probe set comprising a plurality of probes, wherein (i) the probes in the set are capable of detecting an expression level of at least one target selected from Table 3 and/or Table 4; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBT. In an embodiment of the disclosure, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from bladder cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present disclosure may range in length from about 15 nucleotides to the full length of the coding target or non-coding target. In an embodiment of the disclosure, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In an embodiment, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In an embodiment, the probes are at least 15 nucleotides in length. In an embodiment, the probes are at least 15 nucleotides in length. In an embodiment, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In an embodiment, the probe set comprise a plurality of target sequences that hybridize to at least about 5 coding targets and/or non-coding targets selected from Table 3 and/or Table 4. Alternatively, the probe set comprise a plurality of target sequences that hybridize to at least about 10 coding targets and/or non-coding targets selected from Table 3 and/or Table 4. In an embodiment, the probe set comprise a plurality of target sequences that hybridize to at least about 15 coding targets and/or non-coding targets selected from Table 3 and/or Table 4. In an embodiment, the probe set comprise a plurality of target sequences that hybridize to at least about 20 coding targets and/or non-coding targets selected from Table 3 and/or Table 4. In an embodiment, the probe set comprise a plurality of target sequences that hybridize to at least about 30 coding targets and/or non-coding targets selected from Table 3 and/or Table 4. In an embodiment, the probe set comprise a plurality of target sequences that hybridize to at least about 40 coding targets and/or non-coding targets selected from Table 3 and/or Table 4. In an embodiment, the probe set comprise a plurality of target sequences that hybridize to at least about 50 coding targets and/or non-coding targets selected from Table 3 and/or Table 4. In an embodiment, the probe set comprise a plurality of target sequences that hybridize to at least about 60 coding targets and/or non-coding targets selected from Table 3 and/or Table 4. In an embodiment, the probe set comprise a plurality of target sequences that hybridize to at least about 70 coding targets and/or non-coding targets selected from Table 3 and/or Table 4. In an embodiment, the probe set comprise a plurality of target sequences that hybridize to at least about 80 coding targets and/or non-coding targets selected from Table 3 and/or Table 4. In an embodiment, the probe set comprise a plurality of target sequences that hybridize to at least about 90 coding targets and/or non-coding targets selected from Table 3 and/or Table 4. In an embodiment, the probe set comprise a plurality of target sequences that hybridize to at least about 100 coding targets and/or non-coding targets selected from Table 3 and/or Table 4.

The system of the present disclosure further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the disclosure, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In an embodiment, the primers or primer pairs, when used in an amplification reaction, specifically amplify at least a portion of a nucleic acid sequence of a target selected from Table 3 and/or Table 4 (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In embodiments of multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In an embodiment, polynucleotides of the disclosure comprise at least 20 consecutive bases of the nucleic acid sequence of a target selected from Table 3 and/or Table 4 or a complement thereto. The polynucleotides may comprise at least 21, 22, 23, 24, 25, 27, 30, 32, 35, 40, 45, 50, or more consecutive bases of the nucleic acids sequence of a target selected from Table 3 and/or Table 4, as applicable.

The polynucleotides may be provided in a variety of formats, including as solids, in solution, or in an array. The polynucleotides may optionally comprise one or more labels, which may be chemically and/or enzymatically incorporated into the polynucleotide.

In an embodiment, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

A biological sample is collected from a subject in need of treatment for cancer to evaluate whether a patient will benefit from neoadjuvant chemotherapy. Diagnostic samples for use with the systems and in the methods of the present disclosure comprise nucleic acids suitable for providing RNAs expression information. In principle, the biological sample from which the expressed RNA is obtained and analyzed for target sequence expression can be any material suspected of comprising cancerous bladder tissue or cells. The diagnostic sample can be a biological sample used directly in a method of the disclosure. Alternatively, the diagnostic sample can be a sample prepared from a biological sample.

In an embodiment, the sample or portion of the sample comprising or suspected of comprising cancerous tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In an embodiment, the sample is from urine comprising cancerous cells. Alternatively, the sample is from a bladder tumor biopsy.

The samples may be archival samples, having a known and documented medical outcome, or may be samples from current patients whose ultimate medical outcome is not yet known.

In an embodiment, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In an embodiment, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above and elsewhere herein, and those reagents useful for performing the methods described, including amplification reagents, and may include one or more probes, primers or primer pairs, enzymes (including polymerases and ligases), intercalating dyes, labeled probes, and labels that can be incorporated into amplification products.

In an embodiment, the kit comprises primers or primer pairs specific for those subsets and combinations of target sequences described herein. The primers or pairs of primers are suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

In an embodiment, the primers or primer pairs of the kit, when used in an amplification reaction, specifically amplify a non-coding target, coding target, exonic, or non-exonic target described herein, a nucleic acid sequence corresponding to a target selected from Table 3 and/or Table 4, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 3 and/or Table 4, RNA forms thereof, or complements thereto. At least two, three, four or five primers or pairs of primers suitable for selectively amplifying the one or more targets can be provided in kit form. In an embodiment, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the disclosure can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. The mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), and the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, and enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® D Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Sequences

Any method of detecting and/or quantitating the expression of the encoded target sequences can in principle be used in the embodiments disclosed herein. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, serial analysis of gene expression (SAGE), by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for ORT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target sequences and generate target-specific cDNA.

### TaqMan® Gene Expression Analysis

TaqMan® RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present disclosure contemplates that a probe set or probes derived therefrom may be provided in an array format. In the context of the present disclosure, an “array” is a spatially or logically organized collection of polynucleotide probes. An array comprising probes specific for a coding target, non-coding target, or a combination thereof may be used. Alternatively, an array comprising probes specific for two or more of transcripts of a target selected from Table 3 and/or Table 4, or a product derived thereof, can be used. Desirably, an array may be specific for 5, 10, 15, 20, 25, 30, 40, 50, 60, 70, 80, 90, 99 or more of transcripts of a target selected from Table 3 and/or Table 4. Expression of these sequences may be detected alone or in combination with other transcripts. In an embodiment, an array is used which comprises a wide range of sensor probes for bladder-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass: paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In an embodiment of the present disclosure, the Cancer Prognosticarray is a chip.

### Data Analysis

In an embodiment, one or more pattern recognition methods can be used in analyzing the expression level of target sequences. The pattern recognition method can comprise a linear combination of expression levels, or a nonlinear combination of expression levels. In an embodiment, expression measurements for RNA transcripts or combinations of RNA transcript levels are formulated into linear or non-linear models or algorithms (e.g., an ‘expression signature’) and converted into a likelihood score. This likelihood score may indicate the probability that a biological sample is from a patient who will benefit from neoadjuvant chemotherapy. Additionally, a likelihood score may indicate the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm, or variant iterPLIER, or Single-Channel Array Normalization (SCAN) algorithm. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Subtyping

Molecular subtyping is a method of classifying bladder cancer into one of multiple genetically-distinct categories, or subtypes. Each subtype responds differently to different kinds of treatments, and the presence of a particular subtype is predictive of, for example, chemoresistance, higher risk of recurrence, or good or poor prognosis for an individual. The inventors of the present disclosure discovered that a luminal molecular subtype of bladder cancer is clinically useful for predicting patient outcome and response to anti-cancer therapy (see Example 1). As described herein, each subtype has a unique molecular and clinical fingerprint. Differential expression analysis of one or more of the gene targets listed in Table 3 and/or Table 4 allows for the identification of subjects with a luminal subtype of bladder cancer that are at high risk of upstaging to non-organ confined (NOC) disease at radical cystectomy (RC).

### Upstaging to Non-Organ Confined Disease

Bladder cancer of the luminal molecular subtype is associated with lower rates of pathological upstaging from clinical stage T1-T2 to non-organ confined (NOC; ≥pT3 and/or pN+) disease at radical cystectomy (RC). However, approximately one-third of luminal UC were upstaged to NOC disease, and these patients may be undertreated if neoadjuvant chemotherapy (NAC) is withheld. Disclosed herein are genomic classifiers useful for predicting luminal NOC disease in patients diagnosed with clinically organ confined (OC; cT1/T2) disease. In an embodiment, the genomic classifier comprises the gene targets listed in Table 3 and/or Table 4. Genomic classifiers of the disclosure may be used to predict upstaging events within the luminal subtype at TURBT (transurethral resected bladder tumor tissue). As shown in Example 1 below, an exemplary genomic classifier of the disclosure accurately predicted upstaging to NOC (non-organ confined) disease at RC. Since the genomic classifier provides this information at the time of transurethral resected bladder tumor tissue (TURBT), the genomic information provided by the classifier could be used to guide treatment decisions.

### Prognosis and Prediction of Treatment Response to Anti-Cancer Therapy

The genomic classifiers of the disclosure can be utilized to predict outcome and whether or not a bladder cancer patient will benefit from certain anti-cancer therapy (e.g., neoadjuvant chemotherapy). For example, patients with tumors classified as LUC+ have significantly worse outcomes (see Example 1). The LUC (luminal upstage classifier) described in Example 1 below had a 95% sensitivity for identifying lymph node positive disease in bladder cancer patients. Such patients my be undertreated if neoadjuvant chemotherapy (NAC) is withheld. Accordingly, the genomic classifiers of the disclosure are useful for guiding treatment of bladder cancer patients.

### Therapeutic Regimens

Diagnosing, predicting, or monitoring a status or outcome of bladder cancer may comprise treating the bladder cancer or preventing cancer progression. In addition, diagnosing, predicting, or monitoring a status or outcome of bladder cancer may comprise identifying or predicting which patients will be responders or non-responders to an anti-cancer therapy (e.g., neoadjuvant chemotherapy). In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, such as neoadjuvant chemotherapy or other anti-cancer regimen). An anti-cancer regimen may comprise one or more anti-cancer therapies. Examples of anti-cancer therapies include surgery, chemotherapy, radiation therapy, immunotherapy/biological therapy, and photodynamic therapy.

For example, a patient is selected for treatment with neoadjuvant chemotherapy if the patient is identified as being likely to be responsive to neoadjuvant chemotherapy based on expression analysis of the bladder cancer, as described herein. Neoadjuvant chemotherapy may be performed prior to other anti-cancer treatments such as, but not limited to, surgery (e.g., transurethral resection or cystectomy), radiation therapy, immunotherapy (e.g., Bacillus Calmette-Guerin (BCG) or anti-PDL1 immunotherapy), hormonal therapy, biologic therapy, or any combination thereof. Patients, especially those not identified as likely to benefit from neoadjuvant chemotherapy, may omit neoadjuvant chemotherapy and instead be administered other cancer treatments directly.

Examples of chemotherapeutic agents that may be used in treating bladder cancer include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

In an embodiment, the subject has a bladder cancer that is determined to be the luminal molecular subtype. Such subjects could be offered neoadjuvant chemotherapy.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Other chemotherapeutic agents include podophyllotoxin. Podophyllotoxin is a plant-derived compound that may help with digestion and may be used to produce cytostatic drugs such as etoposide and teniposide. They may prevent the cell from entering the GI phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). In an embodiment, the anti-cancer treatment may comprise an FGFR3-inhibitor. Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a bladder tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO₂) lasers, argon lasers, and neodymium:yttrium-aluminum-garnet (Nd:YAG) lasers. Each of these can shrink or destroy tumors and can be used with endoscopes. CO₂ and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

Immunotherapy (sometimes called, biological therapy, biotherapy, biologic therapy, or biological response modifier (BRM) therapy) uses the body's immune system, either directly or indirectly, to fight cancer or to lessen the side effects that may be caused by some cancer treatments. Immunotherapies include interferons, interleukins, colony-stimulating factors, monoclonal antibodies, vaccines, immune cell-based therapy, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy.

Another type of immunotherapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing cancer.

Cancer vaccines are another form of immunotherapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat bladder cancer. Cancer vaccines can be used in combination with other anti-cancer therapies.

Immune cell-based therapy is also another form of immunotherapy. Adoptive cell transfer may include the transfer of immune cells such as dendritic cells, T cells (e.g., cytotoxic T cells), or natural killer (NK) cells to activate a cytotoxic response or attack cancer cells in a patient. Autologous immune cell-based therapy involves the transfer of a patient's own immune cells after expansion in vitro.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty or more of the target sequences corresponding to a target selected from Table 3 and/or Table 4, the subsets described herein, or a combination thereof. In an embodiment, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In an embodiment, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above and disclosed elsewhere herein. The articles may also have gene expression profiles digitally recorded therein so that they may be compared with gene expression data from patient samples. Alternatively, the profiles can be recorded in different representational format. A graphical recordation is one such format. Clustering algorithms can assist in the visualization of such data.

### III. Experimental

Below are examples of specific embodiments for carrying out the present disclosure. The examples are offered for illustrative purposes only, and are not intended to limit the scope of the present disclosure in any way.

Efforts have been made to ensure accuracy with respect to numbers used (e.g., amounts, temperatures, etc.), but some experimental error and deviation should, of course, be allowed for.

### EXAMPLES

### Example 1: Development of a Genomic Classifier to Identify Clinically Aggressive Luminal Bladder Tumors

A genomic classifier for identifying clinically aggressive luminal bladder tumor was developed as follows.

The molecular upstaging (MOL) cohort consisted of microarray data from bladder tumor specimens resected transurethral (TURBT) in 206 patients from seven participating institutions (Lotan et al. Eur Urol, 76: 200, 2019). All patients had high grade clinical stage T1-T2N0M0 urothelial carcinoma of the bladder and underwent RC without NAC. In 189 of 206 patients, at least 8 lymph nodes were removed. In the remaining 17 patients 1-7 lymph nodes were removed. Whole transcriptome profiling was performed on formalin-fixed, paraffin-embedded (FFPE) tissue samples in a Clinical Laboratory Improvement Amendments (CLIA)-certified laboratory (Decipher Biosciences, Inc, San Diego, Calif.). The cohort was specifically selected to exclude high risk features for extravesical disease with strict inclusion and exclusion criteria.

Inclusion criteria included the following: cT1-T2N0 disease; Undergone RC within 3 months of diagnosis; Minimum standard pelvic lymph node dissection at RC.

Exclusion criteria included the following. Received any form of neoadjuvant systemic treatment; Prior pelvic radiation; Presence of hydronephrosis; Presence of cancer in a bladder diverticulum (due to the lack of detrusor muscle these tumors are at a higher risk of being stage pT3); Presence of variant histology (i.e. micropapillary) other than mixed urothelial with squamous or glandular differentiation; Presence of lymphovascular invasion (LVI) at TURBT; Presence of carcinoma in situ (cis) at TURBT; The Cancer Genome Atlas (TCGA) and NAC cohorts have been previously described. (Seiler et al. Eur Urol, 72: 544, 2017 and Robertson et al. Cell, 171: 540, 2017.)

Molecular subtyping of tumors from MOL and TCGA cohorts was carried out as follows. The Seiler 2017 genomic subtyping classifier (GSC) was used to stratify the MOL, TCGA and NAC cohorts into basal, luminal, luminal infiltrated and claudin-low mRNA subtypes. (Seiler et al. Eur Urol, 72: 544, 2017.) From the MOL cohort, 100 patients were identified as luminal and were used for downstream model development. The TCGA cohort included 83 luminal tumors, of which 22 were ≤cT2 and were included in downstream analyses. The NAC cohort included 35 luminal cT2N0 cases. The TCGA classifier was downloaded and applied to the MOL cohort only. (Kamoun et al. Eur Urol, 2019.)

Discovery of an Exemplary Luminal Upstage Classifier

The MOL cohort was used to train an exemplary genomic classifier to predict upstaging to NOC disease at RC for tumors of the luminal subtype. (Seiler et al. Eur Urol, 72: 544, 2017.) To ensure the classifier was applicable to multiple RNA expression platforms, initial gene features (n=25,942) were pre-selected based on commonality between the Affymetrix Human Exon 1.0 ST Array (MOL cohort) and Illumina HiSeq platform (TCGA cohort).

The GSC luminal patients in the MOL dataset (n=100) were randomly split into training and testing sets on a 3:1 ratio. After splitting, the distribution of key-variables between training and testing sets was evaluated, ensuring these variables were distributed equally (no significant difference, P>0.05) (Table 1).

TABLE 1
Equal distribution (P > 0.05) of key clinical variables
among training and testing sets for model development.

| Variable | Train (N = 75) | | Test (N = 25) | | P-value |
|---|---|---|---|---|---|
| NOC disease at | | | | | |
| RC | | | | | |
| Yes | 28 | (37%) | 6 | (24%) | |
| No | 47 | (63%) | 19 | (76%) | 0.33 |
| Age - Median | 71 | [63.1-77.6] | 69.0 | [61.0-74.8] | 0.272 |
| [IQR] | | | | | |
| Sex (%) | | | | | |
| Female | 17 | (23%) | 6 | (24%) | |
| Male | 58 | (77%) | 19 | (76%) | 1.00 |
| Clinical tumor | | | | | |
| stage (%) | | | | | |
| cT1 | 36 | (48%) | 11 | (44%) | |
| cT2 | 39 | (52%) | 14 | (56%) | 0.91 |
| Death during | | | | | |
| follow-up | | | | | |
| Yes | 24 | (32%) | 10 | (40%) | |
| No | 51 | (68%) | 15 | (60%) | 0.63 |

The initial gene list was used for differential gene expression analysis within the training set only, comparing luminal NOC to luminal OC tumors. Model features (genes) were selected based on a median fold-change threshold of ±0.03, with a significance threshold of P<0.05, resulting in 191 candidates. The 191 genes were further filtered based on an expression level standard deviation of >0.2, resulting in the final input set of 99 genes. These genes were used for the training of a 100×10-fold cross-validated, ridge/lasso (α=0.5) penalized logistic regression model (R package GLMNET) in the training set only. For this model, input gene features were grouped into coefficients by cutting a dendrogram that was constructed by clustering all input genes, making the model more generalizable for external dataset testing. The classifier was then applied to the testing set.

To select an optimal probability threshold (Pt) for predicting luminal NOC disease, density plots were evaluated in conjunction with the OptimalCutpoints R package. After training and testing, the Luminal Upstaging Classifier (LUC) was locked and was validated for predicting luminal tumor prognosis within the TCGA cohort. The RNA-seq data were normalized by quantile-quantile matching with the expression values in the MOL cohort, using the R package preprocess Core.

### Statistical Analyses

All statistical analyses were performed using R statistical software (R Foundation for Statistical Computing, Vienna, Austria). The primary endpoint was upstaging to NOC (≥pT3 and/or pN+) at RC in tumors of the luminal subtype. The secondary endpoint was overall survival (OS), calculated as the time from the most recent TURBT (MOL cohort) or from RC (TCGA cohort) until the date of death from any cause. Patients with incomplete follow-up were censored at date of last contact. Kaplan-Meier plots were used to visualize outcome data. Patient and tumor characteristics were compared by Pearson's Chi-squared tests with Yates' continuity correction and two-sided Wilcoxon rank-sum tests. Univariable (UVA) and multivariable (MVA) Cox proportional hazard models evaluated the relationship of the model performance with OS, adjusting for clinical variables.

The clinical characteristics of the luminal patients from the MOL cohort (n=100) are provided in Table 2. Of these cases, 34 were upstaged to NOC (≥pT3 and/or pN+) at RC. The median follow-up was 36.9 [IQR: 24.7-68.5] months and the median time from diagnosis to RC was 1.6 [IQR: 1.0-2.3] months.

TABLE 2
Clinical characteristics of luminal urothelial
carcinoma patients in the MOL cohort.

| Variable | Luminal MOL (N = 100) | | | | P-value |
|---|---|---|---|---|---|
| Upstaged to NOC | OC | (N = 66) | NOC | (N = 34) | |
| (≥pT3 and/or N+) | | | | | |
| at RC | | | | | |
| Age - Median | 71.0 | [62.3-76.8] | 69.0 | [63.9-76.0] | 0.97 |
| [IQR] | | | | | |
| Sex (%) | | | | | |
| Female | 15 | (23%) | 8 | (24%) | |
| Male | 51 | (77%) | 26 | (76%) | 1.00 |
| Clinical tumor | | | | | |
| stage (%) | | | | | |
| cT1 | 39 | (59%) | 8 | (24%) | |
| cT2 | 27 | (41%) | 26 | (76%) | 0.002 |
| Time from | 1.61 | [1.07-2.46] | 1.51 | [1.00-2.16] | 0.37 |
| TURBT to RC | | | | | |
| (months) | | | | | |
| Median [IQR] | | | | | |
| Prior intravesical | | | | | |
| therapy | | | | | |
| No | 41 | | 23 | | |
| Yes | 23 | | 10 | | |
| Unknown | 2 | | 1 | | 0.86 |
| Classified as | | | | | |
| LUC+ (%) | | | | | |
| Yes | 6 | (9%) | 32 | (94%) | |
| No | 60 | (91%) | 2 | (6%) | <0.001 |

### Training a Luminal Upstaging Genomic Classifier

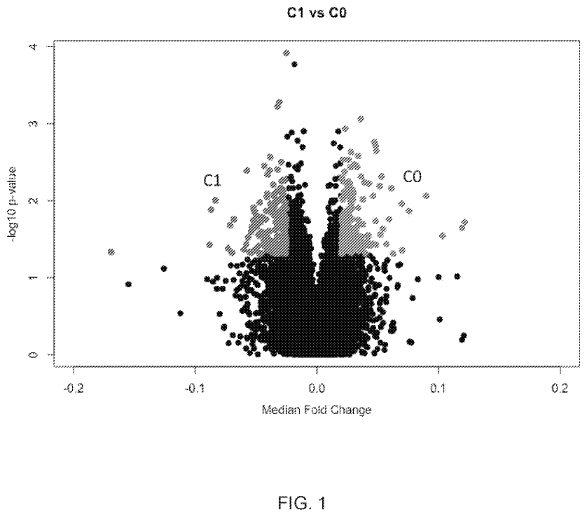

The 100 luminal cases of the MOL cohort were randomly split into training (n=75) and testing (n=25) sets, ensuring there were no significant differences in the distribution of key-variables (see Table 1 above). Differential gene expression analysis within the training set identified 109 upregulated and 82 downregulated genes in luminal NOC tumors (FIG. 1, Table 3). Of these 191 gene features, 99 genes (Table 4) were selected to train a single-sample genomic classifier for predicting luminal NOC tumors (see Methods), this genomic classifier was called the Luminal Upstage Classifier (“LUC”).

TABLE 3
Significant differentially expressed genes in
non-organ confined and organ-confined disease.

OC vs NOC

| Gene | p-value | median fold-change |
|---|---|---|
| ADAM17 | 0.003 | 0.033 |
| APOC1 | 0.032 | 0.062 |
| ARL17A | 0.035 | 0.064 |
| ARL8B | 0.015 | 0.035 |
| ASNS | 0.036 | 0.034 |
| BDH2 | 0.039 | 0.031 |
| BHLHE40 | 0.027 | 0.072 |
| BRK1 | 0.024 | 0.040 |
| C3orf14 | 0.039 | 0.041 |
| C4orf46 | 0.005 | 0.034 |
| CD151 | 0.011 | 0.035 |
| CD9 | 0.022 | 0.052 |
| CERS2 | 0.005 | 0.033 |
| CH17-140K24.5 | 0.033 | 0.042 |
| CISD2 | 0.032 | 0.039 |
| CMTM6 | 0.014 | 0.064 |
| CMTM7 | 0.038 | 0.052 |
| CNN3 | 0.037 | 0.039 |
| CRIPAK | 0.018 | 0.047 |
| CSRP2 | 0.024 | 0.060 |
| CST6 | 0.049 | 0.071 |
| CTB-102L5.4 | 0.043 | 0.056 |
| CTD-2003C8.2 | 0.002 | 0.041 |
| CTD-2547L24.4 | 0.022 | 0.043 |
| CTD-3220F14.1 | 0.022 | 0.044 |
| CYP4Z1 | 0.027 | 0.032 |
| DOCKS | 0.005 | 0.046 |
| ENSA | 0.018 | 0.038 |
| FAM25G | 0.014 | 0.032 |
| FO538757.2 | 0.042 | 0.114 |
| FZD4 | 0.033 | 0.041 |
| GARS | 0.008 | 0.041 |
| GTF3A | 0.030 | 0.039 |
| HAR1B | 0.045 | 0.033 |
| HDAC2 | 0.010 | 0.035 |
| HMGB2 | 0.031 | 0.037 |
| HMGN4 | 0.043 | 0.046 |
| HNRNPAB | 0.044 | 0.053 |
| KCTD11 | 0.007 | 0.033 |
| KRT14 | 0.016 | 0.035 |
| KRT17 | 0.024 | 0.083 |
| KRTAP13-3 | 0.025 | 0.052 |
| LINC00960 | 0.015 | 0.033 |
| MCL1 | 0.024 | 0.035 |
| MKRN2OS | 0.042 | 0.040 |
| MLLT11 | 0.016 | 0.075 |
| MTERF3 | 0.017 | 0.032 |
| MTMR11 | 0.028 | 0.040 |
| MYO10 | 0.001 | 0.031 |
| NAA50 | 0.037 | 0.036 |
| OR10G9 | 0.000 | 0.043 |
| P2RY2 | 0.043 | 0.048 |
| PDIA6 | 0.016 | 0.043 |
| POLB | 0.019 | 0.047 |
| PPAPDC2 | 0.009 | 0.031 |
| PRDX1 | 0.046 | 0.052 |
| PSMD4 | 0.017 | 0.032 |
| PVRIG | 0.030 | 0.031 |
| RARRES1 | 0.022 | 0.179 |
| RP11-105N13.4 | 0.031 | 0.034 |
| RP11-1082L8.3 | 0.003 | 0.050 |
| RP11-115H18.1 | 0.048 | 0.033 |
| RP11-145M4.3 | 0.002 | 0.031 |
| RP11-168F9.2 | 0.028 | 0.085 |
| RP11-247A12.7 | 0.019 | 0.050 |
| RP11-285F16.1 | 0.017 | 0.030 |
| RP11-38L15.8 | 0.005 | 0.119 |
| RP11-462G22.2 | 0.038 | 0.031 |
| RP11-468N14.13 | 0.000 | 0.051 |
| RP11-484P15.1 | 0.039 | 0.060 |
| RP11-539L10.3 | 0.039 | 0.040 |
| RP11-74E22.6 | 0.000 | 0.044 |
| RP11-876F14.1 | 0.013 | 0.074 |
| RP3-406A7.7 | 0.003 | 0.052 |
| RP5-827L5.2 | 0.027 | 0.060 |
| RP5-855F16.1 | 0.011 | 0.042 |
| SERP1 | 0.018 | 0.032 |
| SF3B5 | 0.033 | 0.046 |
| SFN | 0.018 | 0.040 |
| SH3PXD2B | 0.022 | 0.033 |
| SLC2A1 | 0.043 | 0.068 |
| SLC4A7 | 0.010 | 0.030 |
| SLC6A9 | 0.011 | 0.031 |
| SPG20 | 0.011 | 0.032 |
| SPIDR | 0.049 | 0.030 |
| SPOCK1 | 0.045 | 0.039 |
| SREBF1 | 0.006 | 0.040 |
| ST6GALNAC2 | 0.046 | 0.073 |
| TBC1D2 | 0.012 | 0.038 |
| TCEAL1 | 0.014 | 0.065 |
| TCEAL3 | 0.044 | 0.043 |
| TM4SF1 | 0.024 | 0.121 |
| TMEM40 | 0.014 | 0.049 |
| TRIM16L | 0.014 | 0.042 |
| TRIO | 0.012 | 0.036 |
| UCHL3 | 0.034 | 0.063 |
| VDAC3 | 0.031 | 0.046 |
| VHL | 0.034 | 0.041 |
| WBP5 | 0.031 | 0.091 |
| WDR45B | 0.029 | 0.050 |
| WLS | 0.038 | 0.039 |
| YWHAG | 0.009 | 0.048 |
| YWHAQ | 0.009 | 0.040 |
| ZNF256 | 0.018 | 0.051 |
| ZNF260 | 0.027 | 0.053 |
| ZNF662 | 0.004 | 0.059 |
| ZNF680 | 0.020 | 0.045 |
| ZNF717 | 0.001 | 0.047 |
| ZNF790 | 0.004 | 0.040 |
| AC005477.1 | 0.049 | −0.045 |
| AC005614.5 | 0.031 | −0.030 |
| AC011516.1 | 0.049 | −0.036 |
| AC011525.2 | 0.037 | −0.036 |
| AC104653.1 | 0.025 | −0.033 |
| AC112.715.2 | 0.006 | −0.033 |
| ACBD7 | 0.033 | −0.036 |
| AL022578.1 | 0.020 | −0.032 |
| ANP32D | 0.009 | −0.033 |
| CCND2 | 0.001 | −0.035 |
| CGB1 | 0.019 | −0.032 |
| CLC | 0.007 | −0.032 |
| CTA-212A2.3 | 0.033 | −0.038 |
| CTA-276F8.1 | 0.033 | −0.074 |
| CTA-384D8.33 | 0.048 | −0.036 |
| CTA-481E9.3 | 0.019 | −0.046 |
| CTC-264O10.2 | 0.027 | −0.037 |
| CTC-471C19.2 | 0.018 | −0.032 |
| CTD-2034I4.2 | 0.038 | −0.031 |
| FAM102B | 0.010 | −0.040 |
| FAM63B | 0.003 | −0.044 |
| GLCE | 0.007 | −0.031 |
| HIF1A | 0.036 | −0.038 |
| HIGD1C | 0.019 | −0.037 |
| HIST2H2BE | 0.018 | −0.125 |
| HNMT | 0.045 | −0.074 |
| KANTR | 0.029 | −0.038 |
| KIAA1551 | 0.049 | −0.051 |
| LAPTM5 | 0.043 | −0.068 |
| LINC00350 | 0.025 | −0.040 |
| LINC01017 | 0.006 | −0.036 |
| LINC01288 | 0.015 | −0.036 |
| LL22NC03-102D1.18 | 0.028 | −0.043 |
| LLNLR-246C6.1 | 0.034 | −0.033 |
| MGAT2 | 0.011 | −0.040 |
| MRPL20 | 0.039 | −0.034 |
| MYADM | 0.001 | −0.060 |
| NHLRC1 | 0.048 | −0.037 |
| OR10A2 | 0.005 | −0.036 |
| OR13F1 | 0.017 | −0.036 |
| OR13H1 | 0.025 | −0.035 |
| OR52W1 | 0.014 | −0.033 |
| ORC2 | 0.036 | −0.040 |
| PCED1B-AS1 | 0.011 | −0.040 |
| PCNXL4 | 0.033 | −0.030 |
| PRR32 | 0.033 | −0.031 |
| PVALB | 0.024 | −0.031 |
| RP1-90G24.6 | 0.006 | −0.033 |
| RP11-1030E3.1 | 0.042 | −0.033 |
| RP11-164C1.2 | 0.001 | −0.040 |
| RP11-173D3.1 | 0.036 | −0.073 |
| RP11-18C24.8 | 0.003 | −0.053 |
| RP11-23F23.2 | 0.041 | −0.031 |
| RP11-25I9.3 | 0.020 | −0.031 |
| RP11-265E18.1 | 0.003 | −0.041 |
| RP11-265N7.1 | 0.005 | −0.042 |
| RP11-284F21.11 | 0.009 | −0.045 |
| RP11-347C12.10 | 0.045 | −0.034 |
| RP11-353N14.4 | 0.049 | −0.032 |
| RP11-368L12.1 | 0.041 | −0.079 |
| RP11-379L18.3 | 0.008 | −0.047 |
| RP11-381K20.4 | 0.002 | −0.049 |
| RP11-435J9.2 | 0.014 | −0.063 |
| RP11-440I14.2 | 0.045 | −0.040 |
| RP11-44D19.1 | 0.041 | −0.030 |
| RP11-467L19.16 | 0.034 | −0.032 |
| RP11-486I11.2 | 0.036 | −0.037 |
| RP11-554A11.7 | 0.008 | −0.035 |
| RP11-571L19.8 | 0.037 | −0.045 |
| RP11-612B6.1 | 0.008 | −0.054 |
| RP11-63A1.2 | 0.005 | −0.030 |
| RP11-662J14.1 | 0.008 | −0.031 |
| RP11-70F11.7 | 0.038 | −0.048 |
| RP11-789C2.1 | 0.025 | −0.040 |
| RP11-78J21.4 | 0.018 | −0.030 |
| RP11-7F17.4 | 0.006 | −0.031 |
| RP11-810P12.5 | 0.008 | −0.033 |
| RP3-395M20.12 | 0.010 | −0.040 |
| ST20 | 0.018 | −0.036 |
| ST3GAL5-AS1 | 0.013 | −0.031 |
| TAAR2 | 0.045 | −0.031 |
| TBC1D30 | 0.009 | −0.037 |

TABLE 4
Gene features from the Luminal Upstaging Classifier (LUC)

LUC− vs LUC+

| Gene | p-value | Fold Change |
|---|---|---|
| ADAM17 | 0.001 | 0.055 |
| APOC1 | 0.045 | 0.088 |
| ARL17A | 0.010 | 0.113 |
| ARL8B | 0.006 | 0.053 |
| BHLHE40 | 0.002 | 0.124 |
| BRK1 | 0.014 | 0.068 |
| CD9 | 0.062 | 0.053 |
| CERS2 | 0.001 | 0.063 |
| CH17-140K24.5 | 0.033 | 0.066 |
| CISD2 | 0.003 | 0.061 |
| CMTM6 | 0.014 | 0.091 |
| CMTM7 | 0.014 | 0.094 |
| CNN3 | 0.050 | 0.056 |
| CRIPAK | 0.007 | 0.072 |
| CSRP2 | 0.010 | 0.102 |
| CST6 | 0.187 | 0.057 |
| CTB-102L5.4 | 0.065 | 0.065 |
| CTD-2003C8.2 | 0.002 | 0.065 |
| CTD-2547L24.4 | 0.022 | 0.063 |
| CTD-3220F14.1 | 0.014 | 0.066 |
| CYP4Z1 | 0.014 | 0.059 |
| ENSA | 0.006 | 0.058 |
| FO538757.2 | 0.037 | 0.158 |
| GARS | 0.000 | 0.089 |
| GTF3A | 0.041 | 0.052 |
| HMGB2 | 0.004 | 0.070 |
| HNRNPAB | 0.063 | 0.067 |
| KRT14 | 0.062 | 0.044 |
| KRT17 | 0.015 | 0.156 |
| MCL1 | 0.015 | 0.055 |
| MKRN2OS | 0.040 | 0.059 |
| MLLT11 | 0.003 | 0.126 |
| MTERF3 | 0.005 | 0.059 |
| NAA50 | 0.033 | 0.056 |
| OR10G9 | 0.001 | 0.058 |
| PDIA6 | 0.004 | 0.068 |
| POLB | 0.005 | 0.072 |
| PRDX1 | 0.050 | 0.073 |
| PSMD4 | 0.004 | 0.056 |
| RARRES1 | 0.014 | 0.333 |
| RP11-105N13.4 | 0.004 | 0.061 |
| RP11-168F9.2 | 0.083 | 0.119 |
| RP11-247A12.7 | 0.045 | 0.042 |
| RP11-38L15.8 | 0.001 | 0.173 |
| RP11-462G22.2 | 0.087 | 0.042 |
| RP11-539L10.3 | 0.067 | 0.053 |
| RP11-74E22.6 | 0.001 | 0.057 |
| RP11-876F14.1 | 0.104 | 0.094 |
| RP3-406A7.7 | 0.001 | 0.086 |
| RP5-827L5.2 | 0.100 | 0.083 |
| SF3B5 | 0.109 | 0.034 |
| SFN | 0.003 | 0.062 |
| SLC2A1 | 0.004 | 0.124 |
| SPOCK1 | 0.089 | 0.051 |
| ST6GALNAC2 | 0.034 | 0.105 |
| TCEAL1 | 0.008 | 0.119 |
| TCEAL3 | 0.018 | 0.079 |
| TM4SF1 | 0.028 | 0.121 |
| TMEM40 | 0.013 | 0.069 |
| UCHL3 | 0.026 | 0.087 |
| VDAC3 | 0.025 | 0.072 |
| WBP5 | 0.033 | 0.126 |
| WDR45B | 0.002 | 0.105 |
| WLS | 0.010 | 0.067 |
| YWHAG | 0.004 | 0.075 |
| YWHAQ | 0.003 | 0.054 |
| ZNF256 | 0.010 | 0.081 |
| ZNF260 | 0.018 | 0.065 |
| ANP32D | 0.006 | −0.054 |
| CTA-276F8.1 | 0.057 | −0.095 |
| CTA-384D8.33 | 0.047 | −0.056 |
| CTD-2034I4.2 | 0.007 | −0.061 |
| FAM63B | 0.023 | −0.047 |
| HIF1A | 0.222 | −0.023 |
| HIST2H2BE | 0.010 | −0.157 |
| HNMT | 0.035 | −0.117 |
| KIAA1551 | 0.075 | −0.069 |
| LAPTM5 | 0.012 | −0.116 |
| LL22NC03-102D1.18 | 0.075 | −0.060 |
| MRPL20 | 0.210 | −0.019 |
| MYADM | 0.000 | −0.082 |
| OR13H1 | 0.008 | −0.067 |
| OR52W1 | 0.007 | −0.071 |
| PVALB | 0.001 | −0.060 |
| RP1-90G24.6 | 0.008 | −0.053 |
| RP11-173D3.1 | 0.119 | −0.059 |
| RP11-18C24.8 | 0.007 | −0.088 |
| RP11-368L12.1 | 0.039 | −0.145 |
| RP11-379L18.3 | 0.003 | −0.075 |
| RP11-381K20.4 | 0.000 | −0.084 |
| RP11-435J9.2 | 0.005 | −0.106 |
| RP11-440I14.2 | 0.012 | −0.063 |
| RP11-467L19.16 | 0.025 | −0.055 |
| RP11-486I11.2 | 0.063 | −0.050 |
| RP11-554A11.7 | 0.005 | −0.054 |
| RP11-571L19.8 | 0.037 | −0.073 |
| RP11-612B6.1 | 0.001 | −0.093 |
| RP11-789C2.1 | 0.062 | −0.054 |
| ST20 | 0.028 | −0.047 |

### Luminal Upstage Classifier Performance in Training and Testing Set

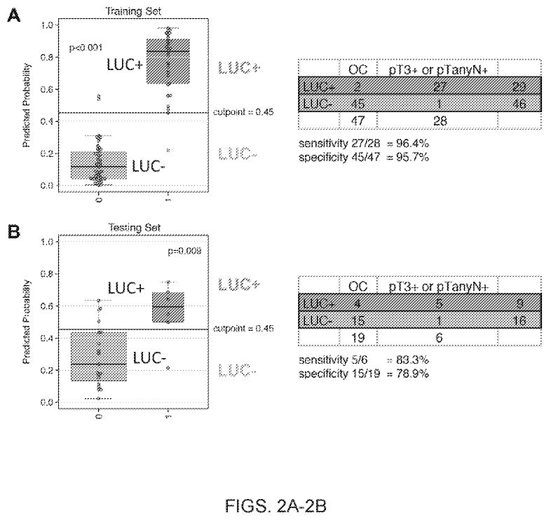

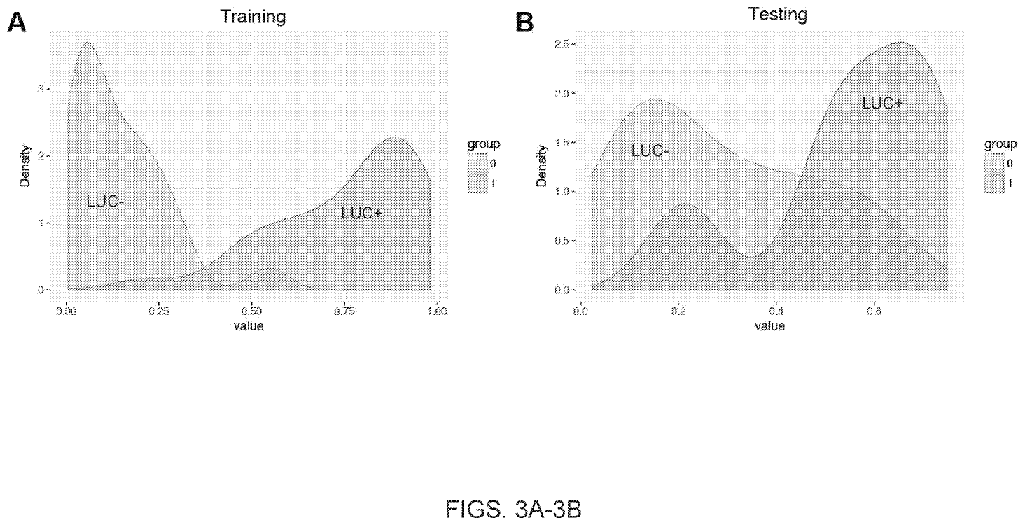

The LUC model was evaluated using discrimination box- and distribution density plots where a probability threshold (Pt) of 0.4529 was selected for classifying positive predicted model cases (LUC+) (FIGS. 2A-2B, FIGS. 3A-3B). Application of the LUC (Pt of 0.4529) to the training set resulted in a sensitivity-specificity combination of 96.4-95.7%, identifying 27/28 NOC cases with two false positives (AUC 0.99). In the testing set, the LUC resulted in an 83.3-78.9% sensitivity-specificity combination, identifying 5/6 NOC cases with four false positive test results (AUC 0.85). Notably, 20/21 pN+ cases were classified as ‘positive’ by the LUC, which corresponds to a 95% sensitivity for identifying lymph node positive disease in the full cohort. Stratifying for clinical stage, the LUC identified 7/8 upstaging cases (88% sensitivity) with 3 false positive test results (36/39=92% specificity) in cT1 tumors. Similarly, the LUC identified 25/26 upstaging cases (96% sensitivity) with 3 false positives (24/27=89% specificity) in cT2 tumors. By the TCGA classifier, the LUC+ cases were classified as luminal papillary (n=33), luminal (n=4) and basal squamous (n=1), while the LUC− cases were luminal papillary (n=53) or luminal (n=9).

Predicted NOC Luminal Tumors have Poor Prognosis

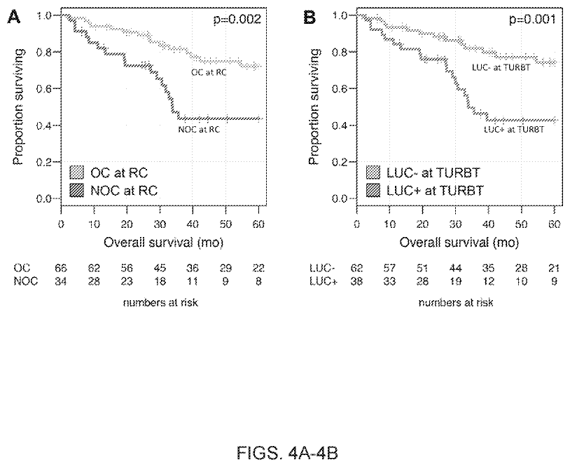

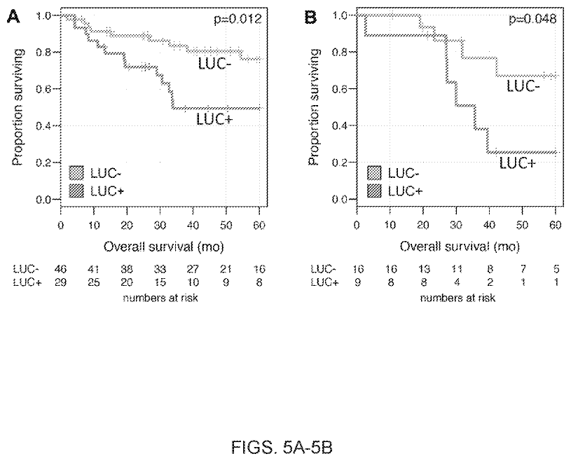

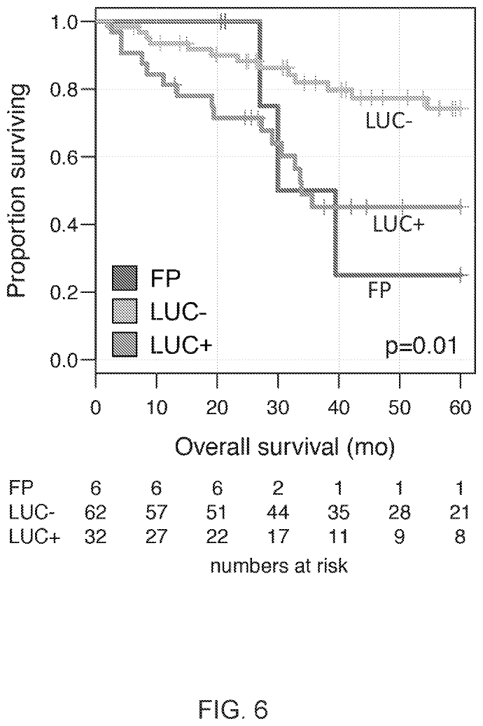

Luminal patients with pathological NOC disease showed significantly worse OS than those with OC disease (P=0.002, Log-Rank test) (FIG. 4A). The performance of the LUC for predicting OS was evaluated and LUC positive (predicted NOC) patients were found to have poor outcomes in both the training and testing sets (FIGS. 5A-5B). In the training set, the 29 LUC positive cases had significantly worse survival than the 46 LUC negative cases (p=0.012). In the testing set, the LUC classified nine patients as ‘positive’, with worse survival (p=0.048), since six out of nine patients died during follow-up. In the full cohort (n=100), the LUC outcome predictions were similar to clinical NOC disease at RC (p=0.001, FIG. 4B). Notably, three of six false positives cases died after 24 months of surgery, indicating an aggressive biological profile may take time to manifest as NOC (FIG. 6). The LUC was also significant for OS on MVA (P=0.004, Table 5), after adjusting for clinical variables available at the time of TURBT.

TABLE 5
Uni- and Multivariable analysis of the variables associated
with overall survival in the luminal MOL cohort (n = 100).

| | Unadjusted HR | | Adjusted HR | |
|---|---|---|---|---|
| Variable | (95% CI) | P-value | (95% CI) | P-value |
| Age | 1.001 | 0.927 | 1.0049 | 0.742 |
| | (0.9723-1.031) | | (0.9761-1.034) | |
| Gender (Male) | 1.328 | 0.504 | 1.2941 | 0.546 |
| | (0.5773-3.056) | | (0.5601-2.990) | |
| cStage (cT2) | 1.4571 | 0.287 | 1.0550 | 0.887 |
| | (0.7291-2.912) | | (0.5050-2.204) | |
| LUC+ | 2.9370 | 0.002 | 2.8848 | 0.004 |
| | (1.479-5.833) | | (1.3929-5.974) | |

LUC Performance within Luminal Madder Cancer Patients from TCGA and NAC Cohorts

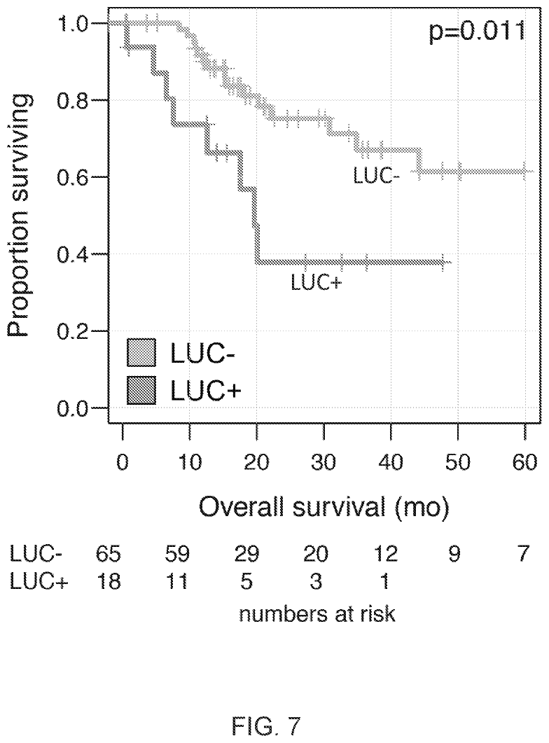

To evaluate the performance of the LUC in additional cohorts, gene expression data was downloaded for a radical cystectomy cohort (TCGA, n=405) and a platinum-based chemotherapy cohort (NAC, n=305). To evaluate the upstaging endpoint in the TCGA dataset, 83 luminal tumors were selected using the GSC (Seiler et al. Eur Urol, 72: 544, 2017), of which 22 were ≤cT2 given the available clinical data. While the GSC was able to predict lower rates of luminal upstaging in the TCGA cohort (Stein et al. J Clin Oncol, 19: 666, 2001), the LUC did not provide additional resolution for this endpoint (Table 6). However, the LUC was prognostic when evaluating OS in the 83 luminal tumors (p=0.011; FIG. 7). On MVA, adjusting for clinical variables available at RC, the LUC remained significant for OS (p=0.043, Table 7).

TABLE 6
Performance of the Luminal Upstaging Classifier
(LUC) for the upstaging endpoint in luminal patients
in the TCGA cohort. (n = 22, cT1-T2).

| Variable | LUC− | LUC+ | Total |
|---|---|---|---|
| No upstaging | 9 | 3 | 12 |
| Upstaging | 5 | 1 | 6 |
| Unknown | 4 | 0 | 4 |

TABLE 7
Uni- and Multivariable analysis of the variables associated
with overall survival in the luminal TCGA cohort (n = 83).

| | Unadjusted HR | | Adjusted HR | |
|---|---|---|---|---|
| Variable | (95% CI) | P-value | (95% CI) | P-value |
| Age | 1.0338 | 0.101 | 1.0543 | 0.057 |
| | (0.9935-1.076) | | (0.9985-1.113) | |
| Gender (Male) | 1.2045 | 0.735 | 1.2098 | 0.743 |
| | (0.41-3.539) | | (0.3878-3.773) | |
| pStage (>pT2) | 2.6795 | 0.062 | 1.5812 | 0.489 |
| | (0.9506-7.553) | | (0.4313-5.797) | |
| pNode (pN+) | 3.3717 | 0.007 | 2.0814 | 0.200 |
| | (1.392-8.168) | | (0.6789-6.381) | |
| LUC+ | 2.911 | 0.015 | 3.2587 | 0.043 |
| | (1.229-6.895) | | (1.0377-10.233) | |

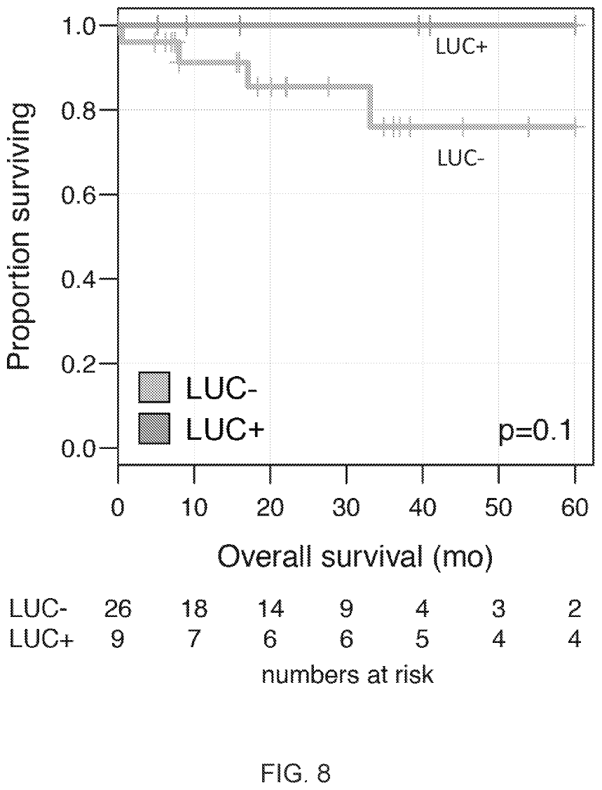

Next, the GSC was used to select 35 luminal cT2NO cases from the NAC cohort (Seiler et al. Eur Urol, 72: 544, 2017), and nine cases were predicted to be positive by the LUC. The primary endpoint of pathological upstaging was not evaluated in this cohort as these were chemotherapy treated patients. However, in the chemotherapy setting, the LUC+ patients were found to have an excellent prognosis and pathological response, with all cases pT2NO or better (FIG. 8, Table 8).

TABLE 8
Pathological response rates in the
NAC cohort stratified by the LUC.

| | pT0 | pTis | pT1 | pT2 | pT3 | pT4 | Total |
|---|---|---|---|---|---|---|---|
| LUC− | | | | | | | 26 |
| pN0 | 8 | 3 | 2 | 6 | 0 | 1 | |
| pN+ | 0 | 0 | 0 | 1 | 2 | 0 | |
| pN = Unknown | 3 | | | | | | |
| LUC+ | | | | | | | 9 |
| pN0 | 3 | 1 | 1 | 2 | 0 | 0 | |
| pN+ | 0 | 0 | 0 | 0 | 0 | 0 | |
| pN = Unknown | 1 | 1 | | | | | |
| Total | 15 | 5 | 3 | 9 | 2 | 1 | 35 |

These results showed that a genomic classifier of the disclosure was useful for identifying aggressive luminal bladder cancer with high rates of upstaging at RC and poor survival. These results further showed that the luminal upstage classifier of the disclosure was useful for identifying lymph not positive disease in patients with bladder cancer. Additionally, these results showed that a genomic classifier of the disclosure was useful for predicting upstaging to non-organ confined (NOC) disease at radical cystectomy (RC). As the genomic classifier was shown to identify bladder cancer patients that had significantly worse outcome at the time of transurethral resected bladder tumor tissue (TURBT), the information from the genomic classifier would be useful for guiding treatment decisions.

These results showed that methods of the present disclosure and genomic classifiers of the disclosure are useful for detecting aggressive luminal bladder cancer. These results further showed that the methods and genomic classifiers of the present disclosure are useful for prognosing or predicting outcome for a subject with luminal bladder cancer. The results suggested that the methods and genomic classifiers of the present disclosure may be used to determine a treatment for a subject with luminal bladder cancer.

What is claimed is:

1. A method comprising:
a) providing a biological sample from a subject having bladder cancer; and
b) detecting the presence or expression level of a plurality of targets in the sample wherein the plurality of targets is selected from Table 3 and/or Table 4.

2. The method of claim 1, further comprising prognosing the bladder cancer according to a genomic classifier based on the expression level of the plurality of targets.

3. A method for determining a treatment for a subject who has bladder cancer, the method comprising:
a) providing a biological sample from the subject;
b) detecting the presence or expression level in the biological sample for a plurality of targets selected from Table 3 and/or Table 4;
c) prognosing the bladder cancer of the subject according to a genomic classifier based on the levels of expression of the plurality of genes; and
d) determining whether or not the subject is likely to be responsive to neoadjuvant chemotherapy based on the expression levels of the plurality of targets in the sample; and
e) prescribing neoadjuvant chemotherapy to the subject if the patient is identified as likely to be responsive to neoadjuvant chemotherapy.

4. A method for treating a subject with bladder cancer, the method comprising:
a) providing a biological sample from a subject having bladder cancer;
b) detecting the presence or expression level in the biological sample for a plurality of targets selected from Table 3 and/or Table 4; and
c) administering a treatment to the subject, wherein the treatment is selected from the group consisting of neoadjuvant chemotherapy or an anti-cancer treatment.

5. The method of any one of claims 1-4, further comprising prognosing the bladder cancer in the subject according to a genomic classifier based on the presence or expression levels of the plurality of targets, wherein said prognosing comprises determining whether or not the subject is likely to have non-organ confined tumors based on the levels of expression of the plurality of targets in the sample.

6. The method of any one of claims 1-5, further comprising prognosing the bladder cancer in the subject according to a genomic classifier based on the presence or expression levels of the plurality of targets, wherein said prognosing comprises determining whether or not the subject is likely to be responsive to neoadjuvant chemotherapy based on the levels of expression of the plurality of genes in the sample.

7. The method of any one of claims 2-6, wherein the prognosing is upstaging to non-organ confined cancer.

8. The method of any one of claims 1-7, further comprising subtyping the bladder cancer based on the expression level of the plurality of targets to a luminal subtype.

9. The method of any one of claims 1-8, wherein the bladder cancer is a luminal subtype.

10. The method of any one of claims 1-9, further comprising determining that the subject has a favorable prognosis if the expression levels of the plurality of targets indicate that the subject will not have non-organ confined tumors or determining that the subject has an unfavorable prognosis if the expression levels of the plurality of targets indicate that the subject will have non-organ confined tumors.

11. The method of any one of claims 1-10, further comprising determining that the subject has a less aggressive tumor if the expression levels of the plurality of targets indicate that the subject will not have non-organ confined tumors or determining that the subject has a more aggressive tumor if the expression levels of the plurality of targets indicate that the subject will have non-organ confined tumors.

12. The method of any one of claims 1-11, further comprising subtyping the bladder cancer based on the expression level of the plurality of targets and administering neoadjuvant chemotherapy to the subject if the subtyping indicates that the subject has the luminal-papillary subtype and administering neoadjuvant chemotherapy to the subject if the subtyping indicates that the subject has the basal/squamous, luminal, luminal-infiltrated, or neuronal subtype.

13. The method of any one of claims 2-12, wherein the neoadjuvant chemotherapy comprises administering cisplatin.

14. The method of any one of claims 4-13, wherein the anti-cancer treatment is selected from the group consisting of surgery, chemotherapy, radiation therapy, immunotherapy, biological therapy, hormonal therapy, and photodynamic therapy.

15. The method of any one of claims 1-13, wherein the method is performed prior to treatment of the patient with anti-cancer therapy.

16. The method of any one of claims 1-14, wherein the biological sample is a biopsy.

17. The method of any one of claims 1-14, wherein the biological sample is a urine sample, a blood sample, or a bladder tumor sample.

18. The method of any one of claims 1-14, wherein the biological sample is transurethral resected bladder tumor tissue.

19. The method of any one of claims 1-18, wherein the subject is a human being.

20. The method of any one of claims 1-19, wherein the level of expression is increased or reduced compared to a control.

21. The method of any one of claims 1-20, wherein said detecting the presence or level of expression comprises performing in situ hybridization, a PCR-based method, a sequencing method, an array-based method, an immunohistochemical method, an RNA assay method, or an immunoassay method.

22. The method of any one of claims 1-21, wherein said detecting the presence or level of expression comprises using a reagent selected from the group consisting of a nucleic acid probe, one or more nucleic acid primers, and an antibody.

23. The method of any one of claims 1-22, wherein said detecting the presence or the level of expression comprises detecting the presence or level of an RNA transcript.

24. The method of any one of claims 1-23, wherein the plurality of targets comprises the 99 genes in Table 4.

25. A kit for prognosing bladder cancer in a subject, the kit comprising agents for detecting the presence or expression levels for a plurality of targets, wherein said plurality of genes comprises one or more targets selected from Table 3 and/or Table 4.

26. The kit of claim 25, wherein said agents comprise reagents for performing in situ hybridization, a PCR-based method, an array-based method, a sequencing method, an immunohistochemical method, an RNA assay method, or an immunoassay method.

27. The kit of any one of claims 25-26, wherein said agents comprise one or more of a microarray, a nucleic acid probe, a nucleic acid primer, or an antibody.

28. The kit of any one of claims 25-27, wherein the kit comprises at least one set of PCR primers capable of amplifying a nucleic acid comprising a sequence of a gene selected from Table 3 and/or Table 4 or its complement.

29. The kit of any one of claims 25-28, wherein the kit comprises at least one probe capable of hybridizing to a nucleic acid comprising a sequence of a gene selected from Table 3 and/or Table 4 or its complement.

30. The kit of any one of claims 25-29, further comprising information, in electronic or paper form, comprising instructions on how to determine if a subject is likely to be responsive to neoadjuvant chemotherapy.

31. The kit of any one of claims 25-30, further comprising one or more control reference samples.

32. A probe set for prognosing bladder cancer in a subject, the probe set comprising a plurality of probes for detecting a plurality of target nucleic acids, wherein the plurality of target nucleic acids comprises one or more gene sequences, or complements thereof, of genes selected from Table 3 and/or Table 4.

33. The probe set of claim 32, wherein at least one probe is detectably labeled.

34. A kit for prognosing bladder cancer comprising the probe set of claim 32 or 33.

35. A system for analyzing a bladder cancer, the system comprising:
a) the probe set of any one of claims 32-33; and
b) a computer model or algorithm for analyzing an expression level or expression profile of the plurality of target nucleic acids hybridized to the plurality of probes in a biological sample from a subject who has bladder cancer and prognosing the bladder cancer of the subject according to a genomic classifier based on the expression level or expression profile of the target nucleic acids in the sample.

36. A kit for prognosing bladder cancer in a subject comprising the system of claim 35.

37. The kit of claim 36, further comprising a computer model or algorithm for designating a treatment modality for the subject.

38. The kit of any one of claims 36-37, further comprising a computer model or algorithm for normalizing the expression level or expression profile of the plurality of target nucleic acids.
